(19) United States
(12) Patent Application Publication
(10) Pub. No.: US 2006/0293296 A1
(43) Pub. Date: Dec. 28, 2006

(54) Process for the preparation of cefpodoxime procetil

(76) Inventors: Milind Moreshwar Gharpure, Maharashtra (IN); Sanjay Shankar Deshmukh, Maharashtra (IN); Asok Kumar Saha, Maharashtra (IN); Rajendra Dagesing Mahale, Maharashtra (IN)

Correspondence Address: COOPER & DUNHAM, LLP, 1185 AVENUE OF THE AMERICAS, NEW YORK, NY, 10036, US

(21) Appl. No.: 10/539,525

(22) PCT Filed: Dec. 20, 2002
(86) PCT No.: PCT/IN02/00240

§ 371 Date: Aug. 18, 2006

(51) Int. Cl. A61K 31/545; C07D 501/14

(52) U.S. Cl. 514/202; 540/222

(57) ABSTRACT

A process for the preparation of cefpodoxime proxetil of high purity conforming to pharmacopoeial specifications.

### FIELD OF INVENTION

The present invention provides a simple industrial method for obtaining cefpodoxime proxetil of high purity conforming to pharmaceutical specifications.

### BACKGROUND OF THE INVENTION

Cefpodoxime Proxetil of formula (I), chemically known as 1-isopropoxycarbonyloxyethyl(6R,7R)-7-[2-(2-aminothiazol-4-yl)-2(Z)-(methoxyimino)acetamido]-3-(methoxymethyl)-3-cephem-4-carboxylate belongs to the third generation of cephalosporin antibiotics, which is administered orally.

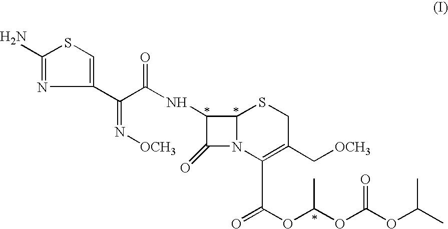

Cefpodoxime proxetil of formula (I) has two asymmetric centers at position 6 and 7 of the cephem nucleus and another one at the α-carbon of 1-isopropoxycarbonyloxyethyl group attached to the 4-carboxyl group as shown in the above structure. The asymmetric center of the α-carbon of 1-isopropoxycarbonyloxyethyl group attached to the 4-carboxyl group of Cefpodoxime proxetil of formula (I) exists as a pair of diastereolsomers known as the R and S isomer. Pharmacopoeial forum Vol: 28 (1), pp 44-52, (2002) mentions that the diastereomeric ratio (R/R+S) of cefpodoxime proxetil should be between 0.5 to 0.6, where the R isomer is less polar among the two stereoisomers.

U.S. Pat. No. 4,486,425 (Nakao, et al) discloses several methods for preparation of cefpodoxime proxetil of formula (I). However, all the methods are associated with formation of varying amounts of impurities. The associated impurities have been removed by taking recourse to chromatographic separation techniques, which albeit provides the compound of formula (I) of desired quality, but is however, cumbersome and not practical on industrial scale.

The impurities which are normally associated with manufacturing processes for cefpodoxime proxetil to name a few, are the Δ² isomer and the E-isomer of cefpodoxime proxetil which have the chemical structures shown below:

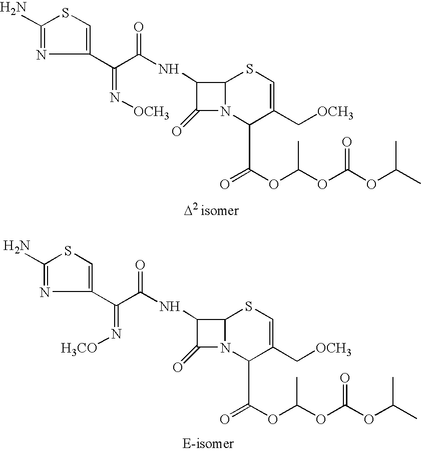

Journal of Antibiotics (Vol: 40, 370, 1987; Fujimoto. K; et. al) describes another method for preparation of cefpodoxime proxetil which consists of the conversion of 7-[2-(2-chloroacetyl amino thiazol-4-yl)-2-(2-methoxyimino acetamido]-3-acetoxy-methyl-3-cephem-4-carboxylic acid (V) into the corresponding 3-methoxymethyl derivative, by reaction with methanol and CaCl₂.2H₂O, which on deprotection of the amino protecting group at the 2-aminothiazolyl ring gives cefpodoxime acid (VI).

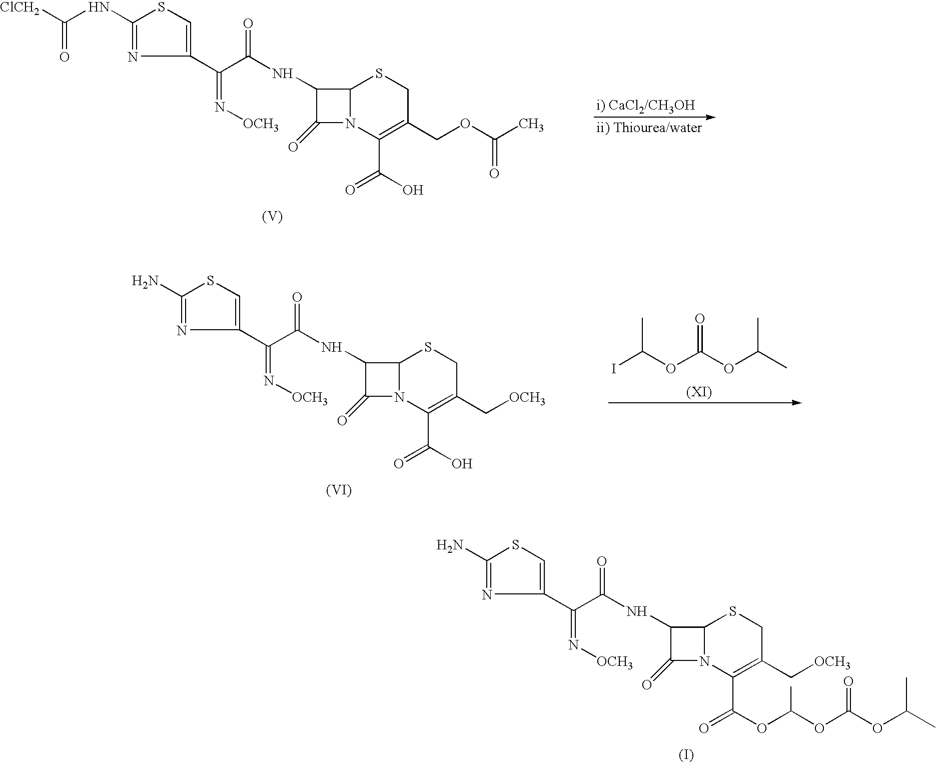

Esterification of the carboxylic acid with 1-iodoethylisopropyl carbonate gives the compound of formula (I).

However, this method also takes recourse to chromatographic methods for obtaining the product in pure form. Moreover, this method also involves additional steps of protection and deprotection of the amino group resulting in overall lower efficiency.

PCT Appln WO 99/35149, A1 (Greil et.al) describes yet another method for preparation of cefpodoxime proxetil of formula (I), wherein, Z-2-(methoxyimino)-2-(2-formylaminothiazol4-yl) acetic acid (VII) activated as the acid chloride hydrochloride on reaction with a compound of formula (VIII), gives N-formylcefpodoxime proxetil of formula (IX).

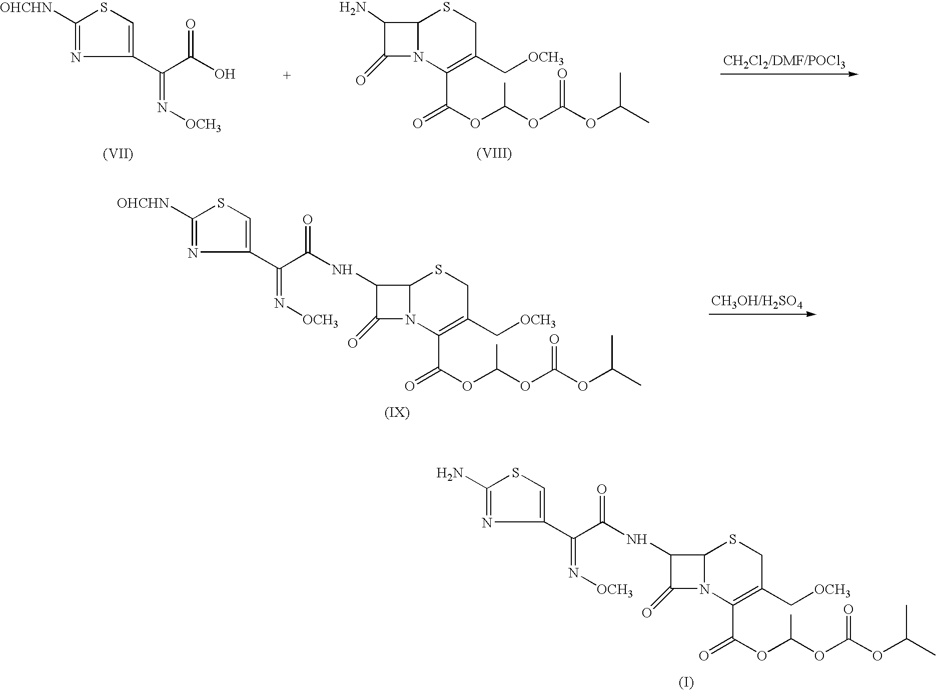

In this method, the intermediate compound of formula (IX) is purified by treating it with water/alcohol mixture containing an additive, such as an organic amide, urea, an imidazolidinone or a pyrimidinone. The purified compound (IX) thus obtained on treatment with acid gives cefpodoxime proxetil of formula (I).

Even though this method does not involve purification, utilizing column chromatography in the final step, additional steps of protection of the amino group as the formyl derivative and its subsequent deprotection are required.

U.S. Pat. application Ser. No. 2002/0065262 A1 (Greil. J, et al ) describes a method for preparation of cefpodoxime proxetil having the right diastereomeric ratio and chemical purity via the intermediacy of a crystalline form of 7-[2-(2-formylaminothiazol-4-yl)-2-(Z)-(methoxyimino)acetamido]-3-methoxymethyl-3-cephem4-carboxylic acid-1-(isopropoxycarbonyloxy)ethyl ester of formula (IX).

The preparation of the crystalline form of n-formyl cefpodoxime proxetil of formula (IX) comprises reaction of 7-amino-3-methoxymethyl-3-cephem-4-carboxylic acid (X) with 1-iodoethyl isopropyl carbonate (XI) to give the compound (VIII), which on condensation with Z-(2-formylaminothiazo-4-yl)-methoxyimino acetic acid activated as an ester or a halogenide gives N-formyl cefpodoxime proxetil which is then crystallized from a mixture of a nitrile or a ketone with water to get crystalline N-formyl cefpodoxime of formula (IX) having a diastereomeric ratio between 0.5 to 0.6.

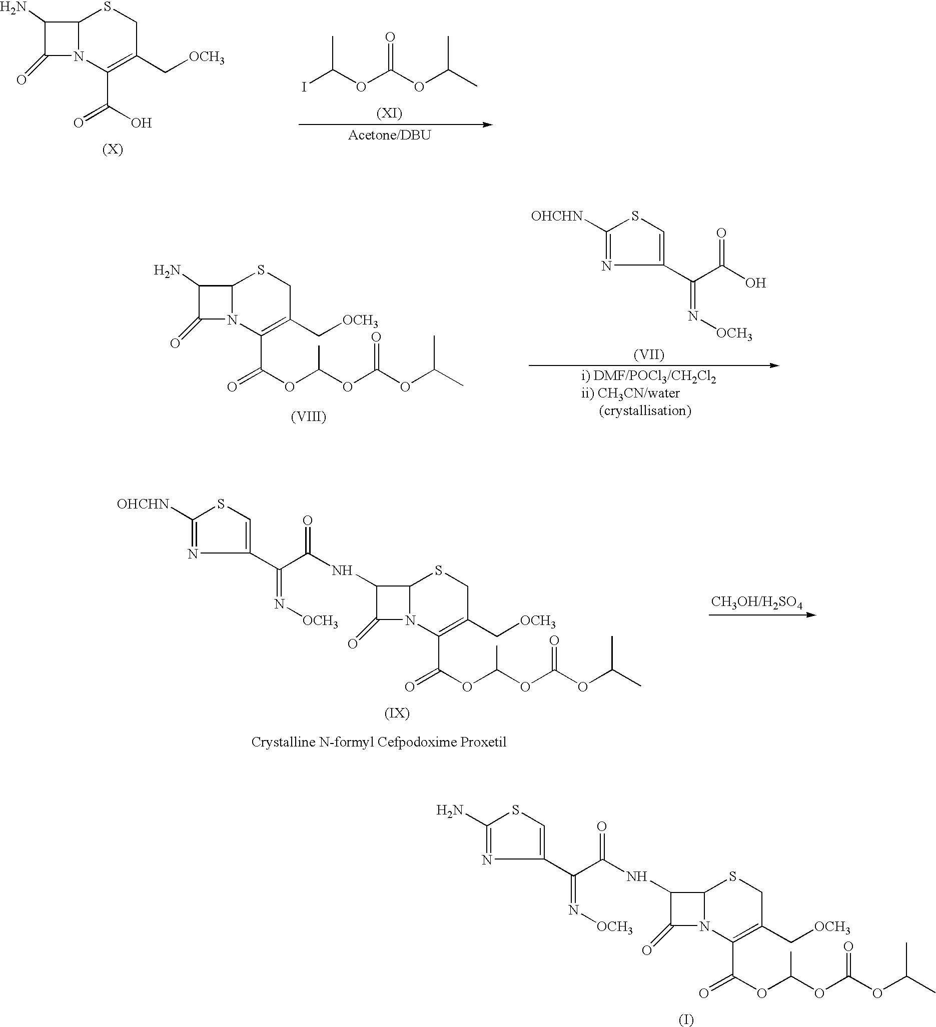

The compound (IX) is then treated with H₂SO₄/methanol to get cefpodoxime proxetil having isomeric ratio between, 0.5 to 0.6.

This method also requires a protection and a deprotection step in addition to a crystallisation step, to give cefpodoxime proxetil, which decreases the overall yield.

PCT Appln WO 02068429 Al (Yatendra Kumar, et al) discloses a method for purification and isolation of cefpodoxime proxetil, obtained by reacting cefpodoxime acid of formula (VI), with 1-iodoethyl isopropylcarbonate of formula (I) in the presence of a base.

The purification of impure cefpodoxime proxetil thus obtained is carried out in two stages which comprises of:
i) dissolving impure cefpodoxime proxetil or adding a solution containing cefpodoxime proxetil into a polar organic solvent or mixture(s) thereof, optionally reducing the solvent by concentration, and adding into a non-polar organic solvent or mixture(s) thereof to precipitate the solid; and
ii) dissolving the solid obtained from the above step into a water miscible polar organic solvent, optionally reducing the solvent by concentration, and adding into water to obtain the pure cefpodoxime proxetil.

However, this method involves a two-step isolation and purification, which moreover involves use of a combination of solvents for crystallization, rendering the method tedious and not commercially attractive.

To summarize, cefpodoxime proxetil prepared by various methods as mentioned herein earlier, is invariably contaminated with varying amounts of impurities associated with the respective method of preparation.

The methods employed for purification of cefpodoximne proxetil utilizes,
i) separation techniques like chromatography,
ii) protection, deprotection of reactive functional groups,
iii) multi-step crystallization,
iv) use of combination of solvents for crystallization,
   which renders such methods less suitable for industrial application.

Therefore, a need exists for a simple, cost-effective method for obtaining cefpodoxime proxetil of high purity, which overcomes the shortcomings of the prior art methods. Such a need has been met by the present invention, which is described in detail herein below.

### OBJECTS OF THE INVENTION

It is an object of the present invention to provide a method for preparation of cefpodoxime proxetil of high purity.

Another object of the present invention is to provide a method for preparation of cefpodoxime proxetil of high purity, which is simple and cost-effective.

Yet another object of the present invention is obtain cefpodoximne proxetil of high purity comprising formation of an acid addition salt of impure cefpodoxime proxetil and its crystallization from an organic solvent.

Yet, further object relates to the preparation of the acid addition salt, which is free from impurities.

A further object relates to the preparation of cefpodoxime proxetil of high-purity and conforming to pharmacopoeial specification from cefpodoxime proxetil acid addition salt comprising neutralization of the acid addition salt by conventional methods.

### SUMMARY OF THE INVENTION

The above and the other objects of the present invention are achieved by the process of the present invention which comprises:
a) adding hydrogen halide to a solution of impure cefpodoxime proxetil in an organic solvent and isolating the hydrohalide salt of cefpodoxime proxetil thus formed, and 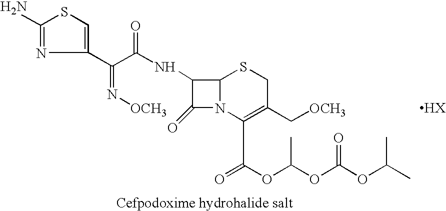
b) dissolving the cefpodoxime proxetil hydrohalide salt obtained in the above step in a water-miscible or water-immiscible organic solvent and neutralizing the salt thus formed with a base followed by isolation of cefpodoxime proxetil in pure form.

In an embodiment of the invention, said water miscible organic solvent is selected from an alcohol, tetrahydrofuran and acetonitrile.

The alcohol is preferably selected from methanol, ethanol, n-propanol, isopropanol, n-butanol, isobutyl alcohol, tertiary butanol.

In a preferred embodiment, said water immiscible solvent is selected from a ketonic solvent, ethyl acetate, methyl isobutyl ketone, chloroform, dichloromethane and 1,2-dichloroethane.

The solvent is preferably a ketonic solvent selected from acetone, methyl ethyl ketone and methyl isobutyl ketone.

In an embodiment of the invention, said ketonic solvent is employed in a volume of from 2.0 to 7.0 times the weight of the impure cefpodoxime proxetil.

The hydrohalide is selected preferably hydrochloric acid, bydrobromic acid and hydroiodic acid.

In a preferred embodiment, the molar ratio of the hydrogen halide used is 1.0 to 1.5 times of cefpodoxime proxetil.

In an embodiment of the invention, said hydrohalide salt is isolated by filtration.

In a preferred embodiment, said pure cefpodoxime proxetil has a diastereomeric ratio between 0.50 and 0.60.

### DETAILED DESCRIPTION OF THE INVENTION

The method for preparation of cefpodoxime proxetil of high purity as per the present invention comprises of the following steps; as summarized in scheme-I.

The first step consists of reaction of cefpodoxime acid of formula (VI) with 1-iodoethyl isopropylcarbonate of formula (XI), to give cefpodoxime proxetil of formula (I).

Synthesis of cefpodoxime proxetil of formula (I):

Typically, cefpodoxime acid of formula (VI) (prepared by any of the general methods reported in U.S. Pat. Ser. No. 4,486,425) is dissolved in any of the solvents like N,N-dimethyl acetamide, N,N-dimethyl formamide, dimethyl sulphoxide but preferably N,N-dimethyl acetamide which is in a ratio of 5-10 times volume per gram of cefpodoxime acid taken.

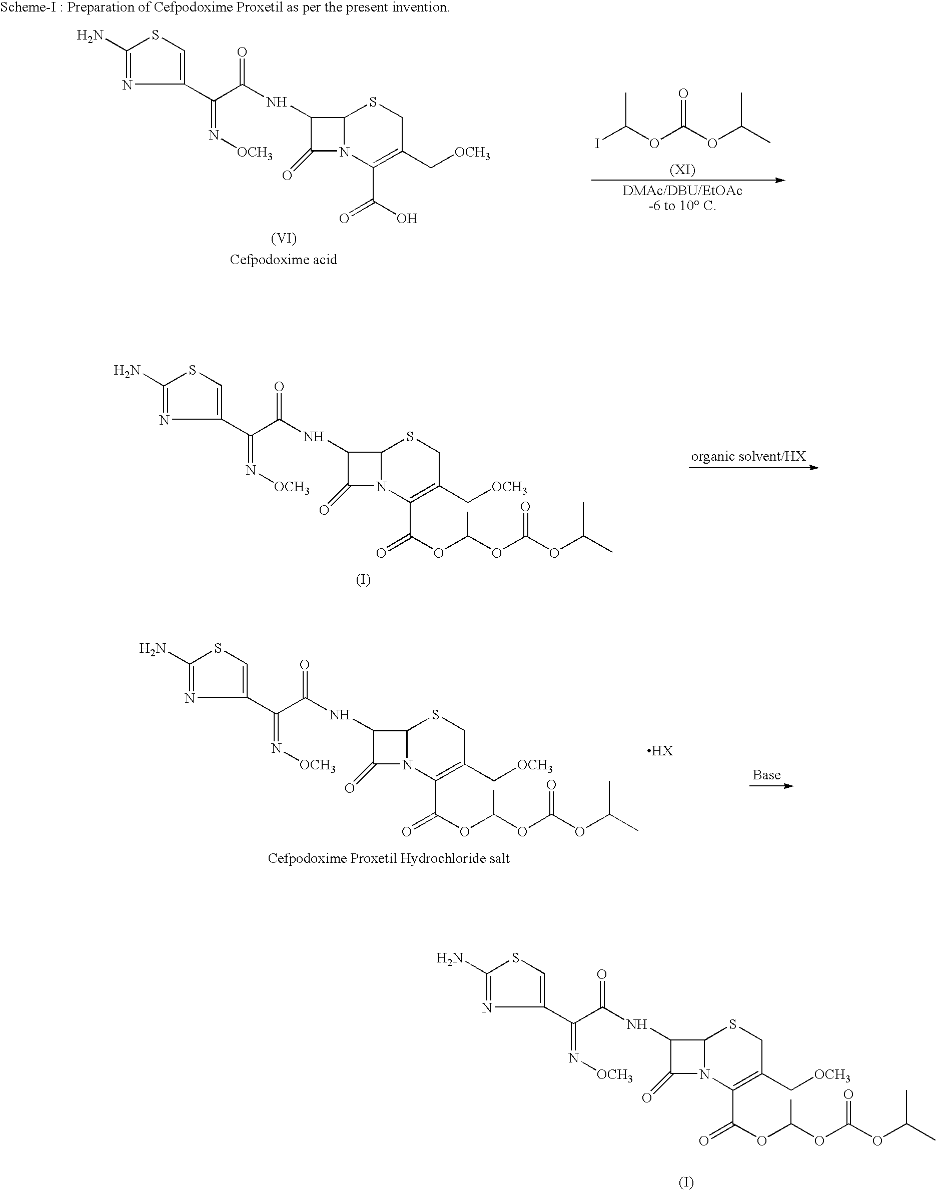

The acid is converted to its salt by using a base selected from a group of inorganic bases such as potassium carbonate, sodium carbonate and sodium bicarbonate preferably sodium carbonate, or from a group of organic bases consisting of dicyclohexyl amine, 1,8-diazabicyclo [5,4,0] undec-7-ene (DBU) preferably the latter, or a mixture thereof

The molar ratio of 1,8-diazabicyclo [5,4,0] undec-7-ene (DBU) or sodium carbonate used, being in the range of 0.95 to 1.02 mole equivalent, but preferably 0.96 to 0.98 mole equivalent with respect to the acid used.

The reaction temperature employed during the salt formation should be between −20° to 0° C., but preferably between −5° C. to −10° C.

1-lodoethylisopropyl carbonate of formula (XI) is then added to the salt of the carboxylic acid at a temperature ranging between −20° C. to 0° C., but preferably between −5° C. to −10° C. The molar ratio of 1-Iodoethylisopropyl carbonate used being in the range of 0.96 to 1.20 moles.

The duration of addition of 1-iodoethylisopropyl carbonate of formula (XI) can be between 5 to 30 minutes but preferably between 5 to 15 minutes.

The reaction temperature employed can be between −20° C. to 0° C., but preferably between −5° C. to −10° C., and the reaction time between 10 to 60 minutes but preferably between 20 to 30 minutes.

Reaction mixture is quenched with a dilute inorganic acid like sulphuric acid, hydrochloric acid but preferably dilute hydrochloric acid.

The quenched reaction mixture is then extracted with an organic solvent like alkyl acetates, which includes methyl acetate, ethyl acetate, and butyl acetate but preferably ethyl acetate.

Organic layer is washed with dilute alkali carbonate solution of alkali carbonates like sodium carbonate, sodium bicarbonate, potassium carbonate but preferably a dilute solution of sodium carbonate. The temperature during the alkali wash should be in the range of −5° C. to +5° C. but preferably between 0° C. to 5° C.

Organic layer is subsequently washed with 5% sodium thiosulphate solution.

Organic layer is optionally treated with carbon and filtered. The filtrate is evaporated under reduced pressure to give an oily residue.

The temperature during the evaporation of the organic solvent can be in the range of 15-35° C. as higher temperature is detrimental for the purity of the compound.

In a specific embodiment, a clear solution of cefpodoxime acid (VI) in dimethyl acetamide is cooled to −5° C. and 1,8-diazabicyclo[5,4,0] undec-7-ene (DBU) in stoichiometric amounts is added in 10 minutes at the same temperature. The reaction mixture is agitated for 5-10 minutes after addition, for complete salt formation after which 1-iodoethylisopropyl carbonate (XI) is added at −6° C. to −10° C. in 10 minutes.

The reaction mixture is agitated for 20 to 30 minutes at −6° C. to −8° C., quenched with 10% hydrochloric acid and the aqueous layer successively extracted with ethyl acetate.

Organic layer is agitated with 2% sodium carbonate solution, at 0-5° C. for 30 minutes and then washed with 5% sodium thiosulphate solution.

Organic layer is optionally treated with activated carbon and filtered. The filtrate is concentrated at reduced pressure and at a temperature not exceeding 35° C. to give a brownish oily residue.

Purification of Cefpodoxime Proxetil (I):Formation of Acid Addition Salt.

The next step comprises of preparation of the acid addition salt of cefpodoxime proxetil by treatment of cefpodoxime proxetil dissolved in a suitable organic solvent with an inorganic acid.

The acid addition salt formation is carried out in an organic solvent. The organic solvents that can be used include alkyl acetates, ethers, chlorinated solvents, ketonic solvents etc. Among all these solvents, ketonic solvents are preferred since the acid addition salt crystallizes out easily and moreover the diastereomeric ratio of the R and S isomer is maintained within permissible limits and also the associated impurities are reduced. The ketonic solvents, which can be used, include acetone, methyl ethyl ketone and methyl isobutyl ketone. Of these ketonic solvents, methyl isobutyl ketone is preferred.

The volume of the ketonic solvent used is between 2.0 to 7.0 times the weight of impure Cefpodoxime proxetil obtained.

Even though various inorganic and organic acids can be used for acid addition salt formation, the salts with inorganic acids are preferred. Of the inorganic acids, addition salts, with hydrohalic acids are preferred since such salts crystallize out easily. The hydrohalic acids that can be used include hydrochloric acid, hydrobromic acid and hydroiodic acid but hydrochloric acid is preferred.

The hydrohalide salts can be prepared by adding an aqueous solution of the corresponding hydrohalic acid to a solution of impure Cefpodoxime proxetil in a ketonic solvent. The salts can also be prepared by bubbling the respective hydrohalide gas into a solution of impure cefpodoxime proxetil in the ketonic solvent.

The molar range of the acid used, is in the range of 1.0 to 1.2 moles per mole of cefpodoxime proxetil.

The acid addition salt formation can be carried out in the temperature range of 0 to 40° C., but preferably between 25° C. to 30° C.

The time required for formation of the acid addition salt can be in the range of 1 to 6 hours, but preferably 2 to 5 hours.

In a specific embodiment, the impure cefpodoxime proxetil of formula (I), obtained in the first part of the synthesis is diluted with methyl isobutyl ketone and stirred at 25° C. to completely dissolve the residue.

Concentrated hydrochloric acid is added in 5-10 minutes and the resulting mixture is stirred for 2.0 hours for complete precipitation of cefpodoxime proxetil hydrochloride salt. Filtration of the hydrochloride salt gives cefpodoxime proxetil hydrochloride, which can be dried-or used as such without drying, for the next step.

Alternatively, the reaction mixture in the first step can be extracted with methyl isobutyl ketone, and after washing with water and sodium thiosulphate solution, the organic layer can be used as such for hydrohalide salt formation, with hydrohalic acid. The volume of the ketonic solvent in the extract is brought in the range of 2.0 to 7.0 times the weight of the impure cefpodoxime proxetil by partial evaporation of the solvent.

The diastereomeric ratio of the R & S isomer present in the hydrohalide salt is in the range of 0.50 to 0.60 which conforms to the specified range of 0.5 to 0.6 mentioned in Pharmacopoeial Forum Vol. 28 (1), pp 44-52, (2002).

The hydrochloride salt is found to be relatively free from all associated impurities and most important the ratio of the R and S isomer is found to remain intact.

Neutralisation of Cefpodoxime Proxetil Hydrohalide Salt.

The next step comprises of neutralizing cefpodoxime proxetil hydrochloride with a base in a water miscible or water immiscible solvent and isolating cefpodoxime proxetil of high purity, which conforms to pharmacopoeial specification.

The cefpodoxime proxetil hydrohalide salt either dried or wet as obtained in the second step can be dissolved in a water miscible solvent and mixed with an aqueous solution of a base. Pure cefpodoxime proxetil is isolated by addition of water. The water miscible solvents include alcoholic solvents, tetrahydrofuran and the nitrites. The alcoholic solvents includes ethanol, methanol but preferably methanol and the nitrites include acetonitrile.

Alternatively, the compound is dissolved in a water immiscible solvent like ethyl acetate, methyl isobutyl ketone, chloroform, dichloromethane, 1,2-dichloroethane but preferably ethyl acetate, and agitated with the solution of a mild base, for complete neutralization of the hydrohalide salt. The organic layer is separated and the solvent can be concentrated to give pure cefpodoxime proxetil.

The base used for liberating the free base viz. cefpodoxime proxetil from its hydrochloride salt can be an inorganic base. The inorganic bases used include sodium hydroxide, sodium carbonate, sodium bicarbonate, potassium bicarbonate; Mild bases are preferred and among them, the alkali bicarbonates, which include sodium bicarbonate and potassium bicarbonate, are preferred, as impurity formation is minimised. Among them, sodium bicarbonate is preferred, as it is milder as compared to potassium bicarbonate.

The time required for addition of the mixture containing cefpodoxime proxetil hydrohalide salt to the alkaline solution varies between 15 to 45 minutes but preferably 30 minutes.

The temperature range is between, 15° C. to 40° C. but the preferred range is 25° C. to 30° C.

In a specific embodiment, cefpodoxime proxetil hydrochloride is dissolved in methanol, at 25° C. to 30° C. and is added to a 1% solution of sodium bicarbonate. The reaction mixture is agitated for 60 minutes. The cefpodoxime proxetil separating out is filtered and dried.

The diastereomeric ratio of the R and S isomer is between, 0.50 to 0.60, which conforms to pharmacopoeial specification.

The invention can be further illustrated by the following examples, which, however, should not be construed as limiting the scope of the invention.

### EXAMPLE 1

Preparation of Cefpodoxime Proxetil (I):

Cefpodoxime acid of formula (VI) (50 gms; 0.117 moles) was added to dimethyl acetamide (350 ml) and stirred to get a clear mixture. The mixture was cooled to −6 to −10° C. 1,8-diazabicyclo [5,4,0] undec-7-ene (DBU) (17.4 gms; 0.114 moles) was added isopropyl carbonate (30.18 gms; 0.117 moles) was then added slowly to the reaction mixture in a period of 10 to 15 minutes. The reaction mixture was agitated for a period of 20 to 30 minutes at the same temperature. The reaction mixture was quenched by addition of 13% hydrochloric acid. The reaction mixture was further diluted with water (400 ml) and extracted with ethyl acetate (500 ml). The separated aqueous layer was re-extracted with ethyl acetate (500 ml). The organic layers were combined and stirred with 2% sodium carbonate solution (500 ml) at 0 to 5° C. for 30 minutes. The organic layer was washed with 5% sodium thiosulphate solution (500 ml). Organic layer was treated with charcoal (7.5 gms) at 25° C. for 60 minutes and filtered through celite bed. The filtrate was concentrated at reduced pressure and at a temperature, below 35° C. The residue was carried forward as such to the next step.

Isomer ratio: (R/R+S):0.51

### EXAMPLE 2

a) Preparation of Cefpodoxime Proxetil Hydrochloride using Methyl Isobutyl ketone as Solvent.

Impure cefpodoxime proxetil (10 gms; 0.018 moles) as obtained in example-1 was dissolved in methyl isobutyl ketone (30 ml) and stirred at 25° C. Concentrated hydrochloric acid (1.8 gms; 0.021 moles) was added to the mixture in 5 minutes and stirred for 120 minutes at the same temperature. The reaction mixture was filtered to give cefpodoxime proxetil hydrochloride (9.6 gms, 90% yield).

Isomer ratio: (R/R+S):0.52

b) Preparation of Cefpodoxime Proxetil Hydrochloride using Acetone as Solvent

Impure cefpodoxime proxetil (16.0 gms; 0.029 moles) as obtained in example-1 was dissolved in acetone (32 ml) and stirred at 25° C. Concentrated hydrochloric acid (3.6 gms; 0.034 moles) was added to the mixture in 5 minutes and stirred for 120 minutes at the same temperature. The reaction mixture was filtered to give cefpodoxime proxetil hydrochloride (11.2 gms; 66% yield).

Isomer ratio: (R/R+S):0.54.

### EXAMPLE 3

Preparation of Cefpodoxime Proxetil Hydrobromide using Methylisobutyl Ketone as Solvent

Cefpodoxime proxetil (5 gms; 0.00897) was dissolved in methyl isobutyl ketone (15 ml) and stirred at 25° C. A 49% solution of hydrobromic acid (1.7 gms; 0.011 moles) was added to the mixture in 5 minutes and stirred for 300 minutes at the same temperature. Cooled to −10° C. and stirred for 1.0 hr. The reaction mixture was filtered to give cefpodoxime proxetil hydrobromide (4.5 gms, 79% yield). Isomer ratio: (R/R+S):0.586

### EXAMPLE 4

a) Preparation of Pure Cefpodoxime Proxetil from Cefpodoxime Proxetil Hhydrochloride Salt using Methanol as Solvent

Cefpodoxime proxetil hydrochloride (9.6 gms; 0.016 moles) obtained in example 2a or 2b or 2c was dissolved in methanol (38 ml) at 25-30° C. and added to a solution of sodium bicarbonate (1.5 gms; 0.016 moles) in water (160 ml) in 30 minutes at 25 to 28° C. The mixture was stirred for 60 minutes. The product separating out was filtered and washed with water 2 x 25 ml and dried to give pure cefpodoxime proxetil& (Yield: 7 gms; %yield: 77.7%; diastereomeric ratio; (R/R+S):0.54).

Purity:≧95 %.

b) Preparation of Pure Cefpodoxime Proxetil from Cefpodoxime Proxetil Hydrobromide Salt using Methanol as Solvent.

Cefpodoxime proxetil hydrobromide (10 gms; 0.016 moles) obtained in example 3 was dissolved in methanol (228 ml) at 25-30° C. and added to a solution of sodium bicarbonate (1.55 gms; 0.018 moles) in water (160 ml) in 30 minutes at 25 to 28° C. The mixture was stirred for 60 minutes. The product separating out was filtered and washed with water 2×25 ml and dried to furnish pure cefpodoxime proxetil (6.50 gms, 75.1% yield; diastereonmeric ratio; {R/R+S}: 0.59).

Purity:≧95%.

c) Preparation of Pure Cefpodoxime Proxetil from Cefpodoxime Hydrochloride Salt using Acetonitrile as Solvent

Cefpodoxime proxetil hydrochloride (3.0 gms; 0.00538 moles) obtained in example 2a or 2b or 2c or was dissolved in acetonitrile (21 ml) at 25-30° C. and added to a solution of sodium bicarbonate (0.55 gms; 0.00654 moles) in water (375 ml) in 30 minutes at 25 to 28° C. The mixture was stirred for 60 minutes. The product separating out was filtered and washed with water 2×25 ml and dried to furnish pure cefpodoxime proxetil (1.8 gms, 63.9 % yield; diastereomeric ratio; {R/R+S}0.54); Purity: 92.6%

d) Preparation of Pure Cefpodoxime Proxetil from Cefpodoxime Hydrochloride using Tetrahydrofuran as Solvent

Cefpodoxime proxetil hydrochloride (3.0 gms; 0.0054 moles) obtained in example 2a or 2b or 2c or was dissolved in tetrahydrofuran (24 ml) at 25-30° C. and added to a solution of sodium bicarbonate (0.55 gms; 0.0065 moles) in water (375 ml) in 30 minutes at 25 to 28° C. The mixture was stirred for 60 minutes. The product separating out was filtered and washed with water 2×25 ml and dried to furnish pure cefpodoxime proxetil (1.2 gms, 42.6% yield; diastereomeric ratio; {R/R+S}:0.54).

Purity:>92%

1. A process for the preparation of cefpodoxime proxetil of formula (I), of high purity conforming to pharmacopoeial specifications, 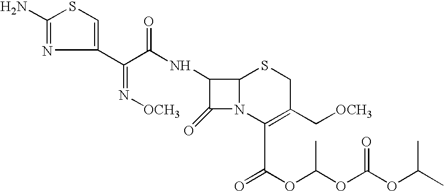
which comprises
a) adding hydrogen halide to a solution of impure cefpodoxime proxetil in an organic solvent and isolating the hydrohalide salt of cefpodoxime proxetil thus formed, and 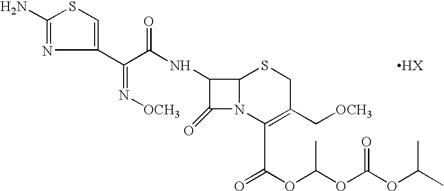
b) dissolving the cefpodoxime proxetil hydrohalide salt obtained in the above step in a water-miscible or water-immiscible organic solvent and neutralizing the salt thus formed with a base followed by isolation of cefpodoxime proxetil in pure form.

2. A process according to claim 1, wherein said water miscible organic solvent is selected from an alcohol, tetrahydrofuran and acetonitrile.

3. A process according to claim 2, wherein said alcohol is selected from methanol, ethanol, n-propanol, isopropanol, n-butanol, isobutyl alcohol, tertiary butanol.

4. A process according to claim 1 wherein said water immiscible solvent is selected from a ketonic solvent, ethyl acetate, methyl isobutyl ketone, chloroform, dichloromethane and 1,2-dichloroethane.

5. A process according to claim 4 wherein said ketonic solvent is selected from acetone, methyl ethyl ketone and methyl isobutyl ketone.

6. A process according to claim 5, wherein said ketonic solvent is employed in a volume of from 2.0 to 7.0 times the weight of the impure cefpodoxime proxetil.

7. A process according to claim 1, wherein said hyrdohalide is selected from hydrochloric acid, hydrobromic acid and hydroiodic acid.

8. A process according to claim 7, wherein the molar ratio of the hydrogen halide used is 1.0 to 1.5 times of cefpodoxime proxetil.

9. A process according to of claim 1, wherein the hydrohalide salt is isolated by filtration.

10. A process according to claim 1, wherein said base is an inorganic base.

11. A process according to claim 10, wherein said inorganic base is selected from sodium bicarbonate, sodium hydroxide, sodium carbonate, potassium carbonate and potassium bicarbonate.

12. A process according to claim 1, wherein the pure cefpodoxime proxetil is isolated by filtration.

13. A process according claim 1, wherein said pure cefpodoxime proxetil has a diastereomeric ratio between 0.50 and 0.60.

14. A process as claimed in claim 1, wherein said treatment of hydrohalide salt of cefpodoxime with said base is carried out in 15 to 45 minutes, preferably, 30 minutes.

15. A process according to claim 1 wherein said treatment with said base is carried out at a temperature of 15 to 40° C., preferably, 25 to 30° C.

16. A process as claimed in any preceding claim wherein after said treatment with said base, said reaction mixture is agitated for 60 minutes.

17. A process according to claim 14 wherein said treatment with said base is carried out at a temperature of 15 to 40° C., preferably, 25 to 30° C.
